(19) United States
(12) Patent Application Publication
(10) Pub. No.: US 2008/0254055 A1
(43) Pub. Date: Oct. 16, 2008

(54) Compositions for Regulation of Hair Growth

(76) Inventors: John Erich Oblong, Loveland, OH (US); Peter B. Styczynski, West Chester, OH (US); James Patrick Henry, Mason, OH (US); Kotikanyadanam Sreekrishna, Mason, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/056,396

(22) Filed: Mar. 27, 2008

Related U.S. Application Data
(60) Provisional application No. 60/922,786, filed on Apr. 11, 2007.

(51) Int. Cl. A61K 36/06; A01N 1/02; A61Q 7/00; A61K 36/87

(52) U.S. Cl. 424/195.16; 435/1.1; 424/766

(57) ABSTRACT

Personal care composition comprising at least one hair growth regulating compound selected from the group consisting of glyceryl dilaurate, apigenin, tetrahydrocurcumin, oleanolic acid, azelaic acid, sulforaphane, canavanine, pyridoxal 5-phosphate, phytic acid, tannic acid, grape seed extract, NG-nitro-L-arginine-methyl ester, benzamidine, sodium butyrate, betulinic acid, polyornithine, polyarginine, fisetin, jasmonates, methyl-jasmonate, cis-jasmone, caffeic acid phenethyl ester, delphinidin, ethyl abietate, esculetin, sorbic acid methyl ester, canaline, N-formyl-methionine, N-formyl-alanine, taurine, palmitoyl carnitine, undecanol, undecylenic acid, rutin, fusidic acid, phenyl pyruvic acid, L-isoleucine, phenyl glycine, silibinin, silymarin, L-ascorbic acid-6-palmitate, N-undecylenoyl-L-phenylalanine, and salts, derivatives and mixtures of any of the foregoing; and a dermatologically-acceptable carrier.

### CROSS REFERENCE TO RELATED APPLICATION

This application claims the benefit of U.S. Provisional Application No. 60/922,786 filed Apr. 11, 2007.

### FIELD OF THE INVENTION

The present invention comprises compositions comprising one or more actives useful for regulating mammalian hair growth, and methods of use thereof.

### BACKGROUND OF THE INVENTION

Hair growth regulation often is desired to improve one's appearance and hygiene. Various methods and personal care products have been developed to remove unwanted hair, for example, shaving, electrolysis, waxing and depilatory creams. However, these methods have several drawbacks. Shaving offers only short term hair removal, and must be repeated. Electrolysis and waxing can keep a treated area free of hair for prolonged periods of time, but can be expensive and/or painful. Additionally, for many consumers, continual growth of hair on the human scalp and face means that one must exert an on-going effort to maintain a desired hair style, for example, cutting and trimming to maintain length, re-coloring to cover the roots of color-treated hair, and/or re-perming to curl the hair that has grown out from the roots. In addition to the cost and inconvenience associated with this on-going hair style maintenance, consumers can also experience a less than desired appearance in between maintenance events, as the hair grows to expose untreated roots and/or to create a longer than desired hair style.

It is therefore desirable to develop methods of regulating hair growth, including retarding, inhibiting, and/or eliminating unwanted hair growth on designated areas of the body, in order to reduce or eliminate the need for repeated hair removal and the drawbacks associated therewith. An additional need exists to provide consumers with a way to maintain a longer lasting hair style appearance, thus reducing the frequency of hair style maintenance needed in order to maintain their hair style.

### SUMMARY OF THE INVENTION

The present invention meets the aforementioned need by providing a method for hair growth regulation comprising applying one or more compounds useful for hair growth regulation, including retarding, inhibiting, or eliminating hair growth to keratinous tissue in need thereof. The compounds may be particularly useful in combination with select chronic skin care actives. Thus, in one embodiment, the composition comprises one or more acute hair growth regulators. The composition additionally may comprise one or more chronic skin care actives, useful for enhancing the benefit(s) of the hair growth regulator and/or for providing at least one additional benefit to the keratinous tissue.

As is known in the art, normal hair growth occurs by a cycle of activity which involves alternating periods of growth and rest. This cycle is often divided into three main stages which are known, in the order in which they occur, as anagen, catagen, and telogen. Anagen is the growth phase of the cycle and may be characterized by penetration of the hair follicle deep into the dermis with rapid proliferation of cells which are differentiating to form hair. Catagen is a transitional stage marked by the cessation of cell division, during which the hair follicle regresses through the dermis and hair growth is ceased. Telogen is often characterized as the resting stage during which the regressed follicle contains a germ with tightly packed dermal papilla cells. During telogen, the initiation of a new anagen phase is caused by rapid cell proliferation in the germ, expansion of the dermal papilla, and elaboration of basement membrane components. This cycle is repeated throughout hair growth. When hair growth ceases, most of the hair follicles remain in the telogen and anagen is not engaged, thus causing the onset of full or partial baldness.

Without being limited by theory, it is believed that certain compounds are able to modulate hair growth by altering protein kinase activity in and surrounding the hair follicular unit in mammalian skin. Protein kinases are key components of signal transduction processes characteristic of the rapid cellular proliferation required for hair growth. For example, compositions comprising apigenin, alone and in combination with other skin care actives, have been found to be useful for regulating mammalian hair growth, including retarding or inhibiting or eliminating hair growth. It is believed that apigenin acts by reducing the activity of mitogen-activated protein (MAP) kinases in the hair follicle and/or surrounding skin. MAP kinases are key components of the transduction of proliferative signals from the cell cytoplasm to the nuclear compartment and are responsible in part for the rapid proliferation required by anagen hair growth. Tetrahydrocurcumin, sulforaphane, azelaic acid, oleanolic acid, caffeic acid phenethyl ester (CAPE), cinnamic acid, ethyl abietate, esculin, sorbic acid methyl ester, cinnamyl anthranilate, tannic acid, grape seed extract are thought to induce cell cycle arrest through multiple mechanisms including, but not limited to, inhibition of cyclooxygenase (COX), inhibiton of carbonic anhydrases, inhibition of proteases—including matrix metalloproteases—and induction of apoptosis.

The following describe some non-limiting embodiments of the present invention.

According to the first embodiment of the present invention, a method of providing hair growth regulation to mammalian keratinous tissue is provided, comprising the step of applying to the keratinous tissue in need of regulation a composition according to the first embodiment. In one embodiment, the benefits are selected from the group consisting of reducing shaving frequency, improving the ease of hair removal, increasing shaving efficiency, making hair softer and finer, making hair less noticeable, slowing the re-growth of hair, reducing erythema and irritation to skin, maintaining a desired hair style for a longer period of time, maintaining a desired facial hair style for a longer period of time, reducing the appearance of pore size, and combinations thereof. Preferably, a method of regulating mammalian hair growth is provided, comprising the step of applying to keratinous tissue in need of hair growth regulation a composition according to the first embodiment.

According to yet another embodiment, a method of providing hair growth regulation to mammalian keratinous tissue is provided comprising: applying to keratinous tissue in need of the benefit a composition comprising at least one hair growth regulating compound selected from the group consisting of glyceryl dilaurate, apigenin, tetrahydrocurcumin, oleanolic acid, azelaic acid, sulforaphane, canavanine, pyridoxal 5-phosphate, phytic acid, tannic acid, grape seed extract, NG-nitro-L-arginine-methyl ester, benzamidine, sodium butyrate, betulinic acid, polyornithine, polyarginine, fisetin, jasmonates, methyl-jasmonate, cis-jasmone, caffeic acid phenethyl ester, delphinidin, ethyl abietate, esculetin, sorbic acid methyl ester, canaline, N-formyl-methionine, N-formyl-alanine, taurine, palmitoyl carnitine, undecanol, undecylenic acid, rutin, fusidic acid, phenyl pyruvic acid, L-isoleucine, phenyl glycine, silibinin, silymarin, L-ascorbic acid-6-palmitate, N-undecylenoyl-L-phenylalanine, and salts, derivatives and mixtures of any of the foregoing; and a dermatologically-acceptable carrier; and delivering energy to the keratinous tissue, wherein the energy is in a form selected from the group consisting of heat, electromagnetic, light, radio-frequency, radio-frequency energy in the absence of light-based energy, mechanical energy, and combinations thereof.

According to yet another embodiment, an article of commerce is provided comprising a composition for regulation of hair growth, and a communication indicating that the composition is useful for providing at least one benefit selected from the group consisting of regulating hair growth, reducing shaving frequency, improving the ease of hair removal, increasing shaving efficiency, making hair softer and finer, making hair less noticeable, slowing the re-growth of hair, reducing erythema and irritation to skin, eliminating the risks of cuts and nicks due to shaving, maintaining a desired hair style for a longer period of time, maintaining a desired hair color for a longer period of time, maintaining a desired facial hair style for a longer period of time, reducing the appearance of pore size, and combinations thereof.

### DETAILED DESCRIPTION OF THE INVENTION

The present invention describes compositions for providing a benefit to mammalian keratinous tissue, and methods of use thereof. The method may comprise the step of delivering energy to the keratinous tissue by means of an energy delivery device. Benefits include, but are not limited to, regulation of hair growth, of bodily malodor, of the appearance of skin and/or of the appearance of hair.

The compositions of the present invention may take a variety of final forms, non-limiting examples of which include lotions, creams, liquids, gels, mousses, emulsions, multi-phase emulsions, solid forms, tonics, shampoos, hair colorants, shave gels (including pre- and post-shave compositions), razor strips, antiperspirants and/or deodorants (including sticks, gels and roll-on liquids), and wipes, and may be applied to the skin via a variety of means. The compositions of the present invention optionally may include additional skin care actives useful for regulating the condition of mammalian skin, skin conditioning agents, perfumes and deodorizers.

Each of the above and additional elements is described herein.

In all embodiments of the present invention, all percentages are by weight of the total composition, unless specifically stated otherwise. All ratios are weight ratios, unless specifically stated otherwise. All ranges are inclusive and combinable. The number of significant digits conveys neither limitations on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

“Keratinous tissue,” as used herein, means keratin-containing layers disposed as the outermost protective covering of mammals and includes, but is not limited to, skin and hair.

“Keratinous tissue in need of regulation,” as used herein, means keratinous tissue exhibiting hair growth, to which it is desired to impart at least one benefit described herein.

“Hair,” as used herein, includes hair on any part of the body.

“Regulating hair growth,” as used herein, means reducing, modulating, inhibiting, attenuating, retarding, and/or diminishing hair growth, and further may include regulating hair appearance, including maintaining a desired hair style for a longer period of time than would be expected without the use of a composition of the present invention. All relative terms used in connection with regulating hair growth and other benefits (such as softer, finer, reducing, less-noticeable, finer, etc.) are understood to mean that the benefit observed is relative to that which is observed or would be expected without the use of a composition described herein.

“Hair growth regulating compound,” as used herein, means a compound useful for regulating hair growth, and is understood not to encompass compounds such as depilatories.

“Derivatives,” as used herein, includes but is not limited to, amide, ether, ester, amino, carboxyl, acetyl, and/or alcohol derivatives of a given compound.

“Isomers,” as used herein, is understood to include D-isomers, L-isomers, and/or DL-isomers of a given compound.

“Salts,” as used herein, includes but is not limited to sodium, potassium, calcium, ammonium, manganese, copper, and/or magnesium salts of a given compound.

“Regulating hair appearance,” as used herein, means that the hair on the skin is visually perceived to be softer, finer, or less noticeable. Additionally, one may notice positive changes in the ease, frequency, and efficacy of hair removal. Reduction and/or inhibition of hair growth may be indicated qualitatively or quantitatively. Qualitative indications of reduction and/or inhibition of hair growth include a person's perception of, for example, softer, finer, or less noticeable hair. Quantitative indications of reduction and/or inhibition of hair growth include, for example, when the mass of the hair removed by shaving is reduced.

“Regulating the condition of mammalian keratinous tissue,” as used herein, means improving appearance and/or feel of keratinous tissue, for example, by providing a smoother appearance and/or feel of skin.

“Improving skin condition” means effecting a visually and/or tactilely perceptible positive change, or benefit, in skin appearance and feel. Benefits that may be provided include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); skin lightening; preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

“Dermatologically-acceptable,” as used herein, means that the compositions or components thereof so described are suitable for use in contact with mammalian keratinous tissue without undue toxicity, incompatibility, instability, allergic response, and the like, is compatible with the actives of the present invention and with the energy delivery device, and will not cause undue safety or toxicity concerns.

“Energy delivery device,” as used herein, means any device used to deliver energy to the skin, hair and other keratinous tissue.

Herein, “delivery of energy,” means that the surface of the keratinous tissue is exposed to the energy emanating from the energy delivery device, where it may penetrate to the desired layers of the tissue, including the hair shaft and/or hair follicle. Herein, “energy” includes but is not limited to energy in the form of light, heat, sound (including ultrasonic waves), electrical energy, magnetic energy, electromagnetic energy (including radiofrequency waves and microwaves), and combinations thereof.

Herein, “continuous level” means that the energy delivered by the device, or energy output, remains at an essentially constant level between the time of device activation and the time of device deactivation.

Herein, “pulsed” means that between the time of device activation and the time of device deactivation, the energy output varies in a predictable manner, characterized by periods of higher output (pulses) alternating with periods of lower output. The onset of pulses typically is sudden as opposed to gradual. “Predictable” means that the pulse peak intensities, pulse shapes, pulse durations, and the temporal spacings between the pulses are substantially identical. The duration of the pulses and the time between pulses may range from on the order of femtoseconds (or 10⁻¹⁵ seconds) to seconds.

Herein, “modulated” means that between the time of device activation and the time of device deactivation, the energy output is characterized by periods of higher output (pulses) alternating with periods of lower output, wherein the pulse intensities, pulse shapes, pulse durations, and the temporal spacings between the pulses may vary significantly. The variation is dependent upon conditions of use, for example the thickness of the skin, hair density, hair coloration, hair thickness, etc. The duration of the pulses and the time between pulses may range from on the order of tenths of a second to tens of seconds.

Herein, “non-modulated” means that between the time of device activation and the time of device deactivation, the energy output is essentially constant, but may be reduced or eliminated under certain circumstances. When the energy output is reduced or eliminated, the device remains activated. Circumstances affecting energy output typically are not directly under control of the operator, but are the result of parameters either programmed or built into the device. These circumstances may include, but are not limited to, lack of contact of the device with the skin, or feedback to the device indicating that a given area of skin already has been treated.

The phrase “treating an/the expanse of skin with an acute hair growth technology,” as used herein, includes shaving, epilating, contacting the skin with a depilatory, waxing, and the like.

“Simultaneously,” as used herein, means that the application of the compositions to the skin and the delivery of energy to the skin essentially are part of a single, continuous action, or alternatively, that the elapsed time between application of the compositions to the skin and the delivery of energy to the skin is minimal. Herein, “minimal” is understood to comprise approximately five minutes or less. An example of minimal elapsed time would comprise the time required to apply the composition to the area of skin requiring application, after which energy would be delivered without further significant delay.

“Sequential” or “sequentially,” as used herein, means that the application of the compositions to the skin occurs prior to and/or after delivery of energy to the skin. Herein, “prior to” means that the first topical application of the compositions to a given area of skin occurs, followed by a period of at least five minutes, and preferably at least 10 minutes, during which no delivery of energy to the skin occurs, followed by a period during which energy is appropriately delivered to the area of skin to which the composition has been applied. Herein, “after” means that energy is appropriately delivered to a given area of skin, followed by a period of at least five minutes, and preferably at least 10 minutes, during which no delivery of energy to the skin occurs, followed by application of the composition to essentially the same area of skin to which energy has been applied.

It is to be understood that “sequentially” encompasses application regimens that alternate application of the compositions to the skin and delivery of energy. The ratio of the number of applications of the composition to the number of times energy is delivered may vary widely, and is within the discretion of those skilled in the art. For example, the compositions may be applied repeatedly and regularly, and energy delivered to the skin less frequently. One example of sequential application would be an application regimen in which the composition is repeatedly administered on a daily or weekly basis, and delivery of energy occurs on a monthly basis.

Herein, “coverage indicator” means any means of indicating to the user that energy has been applied to an area of keratinous tissue. The indication may be in response to a stimulus resulting from, for example, a color change, a chemical change, a change in the level of energy, a change in temperature, etc. Examples of indicators include, but are not limited to, compositions containing a color indicator, an electronic sensor that may emit an auditory or visual signal, or any combination thereof.

Herein, “dietary supplement” means an orally acceptable, dietary ingredient intended to supplement a regular diet, non-limiting examples of which include, vitamins, minerals, herbs or other botanicals, amino acids, enzymes and metabolites. It is to be understood “dietary supplement” includes orally ingestible alpha and/or beta-glucans, wherein the orally-ingestible form comprises a greater concentration of glucans than are found in the unaltered or natural substance from which the glucans are derived. The form in which the dietary supplement is administered may vary widely, and includes, for example, tablets, capsules, gel tablets, and liquids, and may be incorporated into a foodstuff or beverage.

### I. Composition

### A. Hair Growth Regulating Compounds

The topical composition of the present invention comprises from about 0.0001% to about 50%, preferably from about 0.001% to about 10%, and more preferably from about 0.01% to about 5% of at least one hair growth regulating compound selected from the group consisting of glyceryl dilaurate (CAS 27638-00-2), including derivatives such as alpha, alpha-dilaurin (CAS 539-93-5) and 1,2-dilauroyl-SN-glycerol (CAS 61475-84-1)) apigenin (CAS 520-36-5), tetrahydrocurcumin (CAS 36062-04-1), oleanolic acid (CAS 508-02-1), azelaic acid (CAS 123-99-9), sulforaphane (CAS 4478-93-7), canavanine (CAS 543-38-4), pyridoxal-5-phosphate (CAS 41468-25-1; 54-47-7), phytic acid (CAS 83-86-3), tannic acid (CAS 1401-55-4), grape seed extract (CAS 84929-27-1; 20315-25-7; 18206-61-6), L-NAME, (CAS 51298-62-5), benzamidine (CAS 618-39-3), sodium butyrate (CAS 156-54-7), betulinic acid (CAS 472-15-1), polyornithine (CAS 82682-33-5), polyarginine (CAS 26982-20-7), fisetin (CAS 345909-34-4), methyl-jasmonate (CAS 1211-29-6), cis-jasmone (CAS 488-10-8) caffeic acid phenethyl ester (CAS 104594-70-9; 100981-80-4), delphinidin (CAS 528-53-0), ethyl abietate (631-71-0), esculetin (CAS 305-01-1), sorbic acid methyl ester (CAS 1515-80-6), L-canaline (CAS 496-93-5), N-formyl-methionine (CAS 4289-98-9), N-formyl-alanine (CAS 10512-86-4), taurine (CAS 107-35-7), palmitoyl carnitine (CAS 6865-14-1), undecanol (CAS 112-42-5), undecylenic acid (CAS 112-38-9), rutin (CAS 153-18-4), fusidic acid (CAS 6990-06-3), phenyl pyruvic acid (CAS 114-76-1; 156-06-9), L-isoleucine (CAS 73-32-5), phenyl glycine (CAS 875-74-1; 2212-76-2), silibinin (CAS 33889-69-9), silymarin (CAS 84604-20-6), L-ascorbic acid-6-palmitate (CAS 137-66-6), N-undecylenoyl-L-phenylalanine, and salts, derivatives and mixtures of any of the foregoing.

Additionally, the composition of the present invention may comprise from about 0.001% to about 10%, preferably from about 0.01% to about 5%, and more preferably from about 0.1% to about 1% of at least additional compound selected from the group consisting of butylated hydroxytoluene, butylated hydroxyanisole, cetyl pyridinium chloride, a hexamidine compound, a phytosterol such as sitosterol, stigmasterol, campesterol, brassicasterol, kinetin, and zeatin, a green tea catechin compound such as epicatechin, epicatechin gallate and epigallocatechin gallate, salicylic acid, urea, ursolic acid, difluoromethylornithine, alpha-methylomithine, 1,8-diaminooctane, bathocuprione, alanosine, arginine, extracts derived from soy plants, cinnamic acid, cinnamyl anthranilate, mannosamine and salts, derivatives and mixtures of any of the foregoing.

### B. Dermatologically Acceptable Carrier

The compositions of the present invention comprise a dermatologically acceptable carrier for the skin care actives materials. The compositions may comprise from about 50% to about 99.99%, preferably from about 60% to about 99.9%, more preferably from about 70% to about 98%, and still more preferably from about 80% to about 95% of the composition.

The carrier can be a wide variety of types, non-limiting examples of which include solutions, dispersions, emulsions and combinations thereof. Herein, “emulsions” generally contain an aqueous phase and an oil phase. The oils may be derived from animals, plants, or petroleum, may be natural or synthetic, and may include silicone oils. Emulsion carriers include, but are not limited to oil-in-water, water-in-oil and water-in-oil-in-water emulsions. In one embodiment, the carrier comprises an oil-in-water emulsion, a water-in-oil emulsion a silicone-in-water emulsion, and/or a water-in-silicone emulsion. The emulsions may comprise from about 0.01% to about 10%, and preferably from about 0.1% to about 5%, of a nonionic, anionic or cationic emulsifier, and combinations thereof. Suitable emulsifiers are disclosed in, for example, U.S. Pat. No. 3,755,560, U.S. Pat. No. 4,421,769, and “McCutcheon's Detergents and Emulsifiers,” North American Edition, pages 317-324 (1986).

The carrier further may comprise a conditioning agent and/or a thickening agent. Suitable conditioning agents include a humectant, moisturizer, and/or skin conditioner. The composition may comprise from about 0.01% to about 20%, preferably from about 0.1% to about 10%, and more preferably from about 0.5% to about 7%, of a conditioning agent. In one embodiment, the conditioning agent is selected from urea, guanidine, sucrose polyester, panthenol, dexpanthenol, allantoin, glycerol, and mixtures thereof.

The compositions of the present invention may comprise from about 0.05% to about 5%, preferably from about 0.1% to about 4%, and more preferably from about 0.25% to about 3%, of a thickening agent. Suitable classes of thickening agents include, but are not limited to carboxylic acid polymers, crosslinked polyacrylate polymers, polyacrylamide polymers, polysaccharides, gums and mixtures thereof. Non-limiting examples of suitable thickening agents are described in the “CTFA International Cosmetic Ingredient Dictionary,” 10^{th} Ed. (2004), pp. 2294-96.

The composition further may comprise from about 0.1% to about 20%, preferably from about 1% to about 10%, and more preferably from about 5% to about 7% of dipropylene glycol monocaprylate, isopropyl lauroyl sarcosinate, and mixtures thereof, suitable for use with oil soluble actives. Dipropylene glycol monocaprylate is commercially available as Caproyl™ 90 (Gattefosse, Gennevilliers, France). Isopropyl lauroyl sarcosinate is commercially available as Eldew™, which can be purchased from Ajinomoto U.S.A., Paramus, N.J. Non-limiting examples of oil-soluble actives suitable for use with dipropylene glycol monocaprylate and/or isopropyl lauroyl sarcosinate include, but are not limited to, tetrahydrocurcumin, tetrahydrocurmin diacetate, glycyrrhizic acid, glycyrrhetinic acid, ginger extract, ursolic acid, proanthocyanidins, green tea polyphenols and mixtures thereof.

### C. Chronic Skin Care Actives

The composition of the present invention may comprise from about 0.001% to about 10%, preferably from about 0.01% to about 5%, and more preferably from about 0.1% to about 1%, of at least one chronic skin care active, useful for providing at least one additional benefit to mammalian keratinous tissue, including, but not limited to reducing the appearance of wrinkles, deep lines, fine lines, crevices, bumps, large pores; increasing the convolution of the dermal-epidermal border; skin lightening; increasing elasticity, decreasing sagging, reducing cellulite; reducing the appearance of under-eye circles, reducing the appearance of discoloration, reducing hyperpigmentation, increasing skin luminosity, and combinations thereof. Non-limiting examples of suitable chronic skin care actives include polyacrylamide/C13-14 isoparaffin/laureth-7 (commercially available as Sepigel™ 305), niacinamide, N-acetyl glucosamine, polyacrylamide/C13-14 isoparaffin/laureth-7, carnosine, vitamin E acetate, N-acetyl-D-glucosamine, glucosyl hesperidin, ascorbyl glucoside, palmitoyl-lysine-threonine-threonine-lysine-serine, palmitoyl-lysine-threonine, cetyl betaine, bisabolol, theophylline, dihydroxyacetone, panthenol, butyl methoxydibenzoylmethane, glycyrrhizic acid, glycyrrhetinic acid, ergothioneine, octadecenedioic acid, retinyl propionate, yeast extract, and salts, and derivatives and mixtures of any of the foregoing.

### D. Optional Skin Care Actives

The composition of the present invention may comprise at least one additional skin care active, useful for regulating and/or improving the condition of mammalian skin. Non-limiting examples of suitable skin care actives are described in U.S. Patent Application 60/818,152, filed Jun. 30, 2006, and in U.S. Patent Application 2007/0065381A1. In one embodiment, the additional skin care active comprises from about 0.0001% to about 20%, preferably from about 0.001% to about 10%, and more preferably from about 0.01% to about 5% of at least one additional hair growth regulating compound, selected from the group consisting of resveratrol, wortmannin, compounds that decrease Cadherin-11 and TRPS1, Bowman-Birk protease inhibitor, scaffold protein and anti-VEGF (Vascular endothelial growth factor) peptides, TRPV3 (the ion channel Transient Receptor Potential V3) antagonists, protein arginine methyltransferase inhibitors, palmatine, white chrysanthemum petal extract and seed extract of burdock root, RNAi targeted to the human demosglein-4 or nude mRNA, Sophora flavescens extract, Sophora japonica extract Leucojum aestivum, ginseng extract, pineapple extract, koji extract, rose hip extract, apricot extract kiwi extract, angiopoietin-like 4 protein antagonist, borax, toxic albumin proteins from cytotoxic lectins, ricin, abrin and modeccin, malonic acid, malic acid, modulators of semaphorin 3B, equol, urtica, geldanamycin, substance P, and salts, derivatives, and combinations of any of the foregoing. Preferably, the additional hair growth regulating compound is selected from the group consisting of resveratrol, wortmannin, palmatine, malonic acid, malic acid, equol, geldanamycin, and salts, derivatives, and combinations of any of the foregoing.

### II. Energy

The energy delivered to the keratinous tissue includes, but is not limited to, energy in the form of light, heat, sound (including ultrasonic waves), electrical energy, magnetic energy, electromagnetic energy (including radio frequency energy), mechanical energy, and combinations thereof. In one embodiment, the energy is delivered to the keratinous tissue in the form of light, heat, and ultrasonic sources, and combinations thereof. In yet another embodiment, the energy is delivered in the form of light, and preferably, laser-generated light. Mechanical energy may be delivered by means of a mechanical hair removal device (i.e., as an adjunct to hair removal via a blade, razor, epilator, electrical powered or non-electrical shaving apparatus, etc.).

The energy may be delivered from these various sources in a manner that is continuous, pulsed, modulated, non-modulated, and combinations thereof. Preferably, the energy is non-modulated.

### A. Light Energy Sources

“Light energy,” as used herein, means light emitted from laser and/or non-laser light sources. The light energy may be coherent or non-coherent, monochromatic or polychromatic, and collimated, diffuse or divergent. Polychromatic light may be filtered to provide the desired wavelength or a selected band of wavelengths.

Laser light sources include solid-state lasers, gaseous lasers and combinations thereof. Non-limiting examples of solid-state laser light sources include Nd:YAG (Neodymium:Yttrium Aluminum Garnet), ruby and alexandrite. Non-limiting examples of gaseous laser sources include helium-neon, argon, and carbon dioxide. Examples of the use of suitable laser light sources are disclosed in U.S. Pat. Nos. 6,063,074 and 6,152,917, both issued to Tankovich. Additional laser light sources include, but are not limited to, diode lasers, diode laser bars, or diode laser arrays. See, for example, U.S. Pat. No. 6,273,885, issued to Koop et al.

Non-limiting examples of non-laser light sources include flashlamps, halogen lamps, light-emitting diodes (LED's), intense pulsed light (IPL) sources and combinations thereof. The wavelengths may comprise the ultraviolet, visible, near-infrared and infrared regions of the electromagnetic spectrum. Preferably, the wavelength will be in the visible light range. Examples of suitable non-laser light sources are disclosed in U.S. Pat. Nos. 5,885,273, 6,174,325, and 6,280,438, all issued to Eckhouse et al.

In one embodiment, the energy delivery device is of a weight and dimensions suitable for an average adult human to hold comfortably in one hand. Preferably, the volume of the device is 1500 cubic centimeters (ccm) or less, more preferably the volume is less than 1000 ccm, and still more preferably, the volume is less than 750 ccm. Preferably, the weight of the device is 1 kilogram (kg) or less, more preferably the weight is less than 750 grams, and still more preferably the weight is less than 500 grams. An example of a suitable hand-held energy delivery device is described in US Patent Application 2004/0167499 A1 (Grove et al.).

The energy delivery device further may comprise a means for pre-heating or for cooling the skin. See, for example, U.S. Pat. No. 6,273,884, issued to Altshuler et al. Additionally or alternatively, the energy delivery device is cordless.

### B. Heat Energy Sources

The energy delivered to the keratinous tissue may be in the form of heat energy. The keratinous tissue, including portions of the hair within the follicles, may be heated by broad band radiation emitted by the heat source, for example, a flash lamp. Alternatively, the heat may be generated by means of visible radiation, delivered, for example, by a high intensity lamp such as a xenon arc lamp. The heat delivery device may include a means for preventing overheating of the skin, for example, by manually or automatically distancing the apparatus from the skin, by pumping air into region surrounding the skin selected time after the flashing of the flash lamp, or by other suitable means of cooling the skin. Examples of devices that utilize heat energy are disclosed in U.S. Pat. No. 6,187,001, issued to Azar et al.

### C. Ultrasound Energy Sources

The energy delivered to the keratinous tissue may be in the form of ultrasound, or ultrasonic, energy. Ultrasound energy delivery may comprise higher-frequency sound waves that are greater than about 40,000 Hertz (Hz), or alternatively lower frequency sound waves comprising frequencies of about 40,000 Hertz or less. The energy produced by the sound waves may penetrate keratinous tissue, with the depth of penetration dependent upon factors including the acoustic density of the sound waves, the frequency of the sound waves, and the composition of the keratinous tissue. The output energy generally will range from milliwatts to watts.

The delivery of the sound waves may be focused, collimated, diffuse, and combinations thereof. The delivery of the sound waves further may be continuous, pulsed, modulated, non-modulated, and combinations thereof. The ultrasound energy usually is delivered through a transducer head. When used on skin, it is usually placed in direct contact with the skin using a coupling medium, one example of which is an aqueous gel.

Examples of suitable ultrasound devices are disclosed in US Patent Application 2002/0120225 A1, filed by McDaniel on Jun. 8, 2001; U.S. Pat. No. 6,732,744, issued to Olshavsky et al.; and U.S. Pat. No. 6,544,259, issued to Tsaliovich.

### III. Methods of Use

### A. Application of the Compositions

The present invention describes compositions useful for regulating hair growth and methods of use thereof. Additionally or alternatively, the compositions may provide at least one additional benefit to the keratinous tissue, including but not limited to, hair growth regulation, reducing shaving frequency, improving the ease of hair removal, increasing shaving efficiency, making hair softer and finer, making hair less noticeable, slowing the re-growth of hair, reducing erythema and irritation to skin, maintaining a desired hair style for a longer period of time, maintaining a desired hair color for a longer period of time, maintaining a desired facial hair style for a longer period of time, reducing the appearance of pore size, and combinations thereof.

Application of the compositions can occur through a variety of means, including with the fingers or hands, or by using an implement. Non-limiting examples of implements include a pad, cotton ball, applicator pen, roll-on applicator, stick applicator, spray applicator, substrate, razor strip, electronic delivery devices (including a motor-driven applicator, exfoliating applicator, etc.) and patches. The compositions may be secured to and/or enclosed within the energy delivery device itself.

A wide range of quantities of the compositions of the present invention can be employed to improve the condition of the skin. Quantities of the present compositions typically applied per cm² of skin are from about 0.01 mg/cm² to about 10 mg/cm². One useful application amount is about 0.5 mg/cm² to about 5 mg/cm². The composition may be applied to any part of the external portion of keratinous tissue in need of regulation of hair growth and/or any additional benefit described herein.

### B. Delivery of Energy to the Skin

The energy delivery device may deliver energy in a variety of forms, including but not limited to energy in the form of light, heat, sound (including ultrasonic waves), magnetic energy, electromagnetic energy (including radiofrequency waves and microwaves), and combinations thereof. Delivery of energy to the skin means that an effective amount of energy is applied to the keratinous tissue via a means suitable for the type of energy being delivered. The energy from these various sources may be continuous, pulsed, modulated, non-modulated, and combinations thereof. In one embodiment, the energy delivery device is hand-held. Additionally or alternatively, the energy delivery device is cordless.

The energy may be applied by holding the device to a single area of keratinous tissue, and subsequently moving the device to another area of tissue (or “stamping”). Alternatively, the energy may be applied as the device is continuously moved, or scanned, across the surface of the tissue. The scanning velocity will depend upon a variety of factors, examples of which include the size of the device, the amount of hair, the type of keratinous tissue, and the amount of energy delivered. The device may be held in substantially continuous contact with the surface of the keratinous tissue, as with laser devices, or may be held at a short distance from the keratinous tissue with the energy directed toward the surface, as with flash lamps.

In one embodiment, the energy output is non-modulated, with the non-modulation regulated by feedback received by the device. This feedback may result from energy being delivered into the skin, where a change is induced in the skin itself or in some compound applied to the skin. Alternatively, the area of the skin in which a change is induced is similar in size to the area to which the energy delivery device delivers energy to the skin. Alternatively, the area of the skin in which a change is induced is larger than the area to which the energy is delivered to the skin.

A temperature change may be simultaneously induced in the keratinous tissue or alternatively, in a compound applied to the surface of the skin. This temperature change is in addition to any temperature change induced by the delivered energy itself. For example, the keratinous tissue may be heated prior to delivery of energy, or alternatively, the keratinous tissue may be cooled after delivery of energy.

For energy derived from ultraviolet light sources, the wavelength will generally fall within the UV-A range, from about 315-400 nm, where “nm” means 1×10⁻⁹ meters. For energy derived from visible light sources, the wavelength will generally range from about 400 nm to about 700 nm. For energy derived from infrared (IR) light sources, the wavelength will generally range from about 700 nm to about to about 3000 nm. The amount of energy delivered, or “output fluence,” may be in the range of about 1 J/cm² to about 100 J/cm², where “J” means Joules. For pulsed light sources, the pulse length may range from about 0.001 seconds to about 3 seconds, with an average pulse duration of from about 0.001 seconds to about 1 second. The surface area of keratinous tissue to be covered will vary depending on the application. These and other parameters relevant to delivery of energy depend upon the type of treatment and the type of tissue to be treated, and will appropriately be selected by one of skill in the art.

### C. Application Regimens

The compositions of the present invention may be applied to the skin simultaneously and/or sequentially with a mechanical hair removal device (i.e., as an adjunct to hair removal via a blade, razor, epilator, shaving apparatus, etc.), with a chemical hair removal composition (depilatories, waxes, etc.) and/or with delivery of energy to the skin. The method optionally may comprise the step of applying energy to the keratinous tissue and/or removal of hair with a blade, epilator, depilatory, and/or razor.

In one embodiment, the method of use may comprise the step of applying a composition of the present invention to hair on the scalp and optionally styling and/or coloring the hair according to conventional means, to facilitate longer retention of a desired hair style and/or hair color.

### IV. Kit

The present invention further may comprise a kit. The kit may comprise a composition comprising at least one hair growth regulating compound and at least one additional component selected from the group consisting of an energy delivery device, a coverage indicator, a mechanical hair removal device (including a razor, epilator, shaving apparatus, tweezers, etc.), depilatories, waxes, an implement, at least one additional personal care composition, an dietary supplement, instructions for complying with a treatment regimen, instructions for the use of a coverage indicator; and combinations thereof. The kit may contain individual inner packaging units, each containing a quantity of composition suitable for use in a single application regimen. In one example, the individual packaging units will contain 10 ml, preferably 5 ml, more preferably 1 ml, and still more preferably 0.5 ml of a composition described herein.

### V. Article of Commerce

The present invention also provides an article of commerce comprising a personal care composition useful for regulating hair growth, as described herein, and/or for providing at least one additional benefit to the keratinous tissue. The additional benefit may be selected from the group consisting of reducing shaving frequency, improving the ease of hair removal, increasing shaving efficiency, making hair softer and finer, making hair less noticeable, slowing the re-growth of hair, reducing erythema and irritation to skin, eliminating the risks of cuts and nicks due to shaving, maintaining a desired hair style for a longer period of time, maintaining a desired hair color for a longer period of time, maintaining a desired facial hair style for a longer period of time, reducing the appearance of pore size, and combinations thereof. One preferred article of commerce comprises a personal care composition described herein and a communication indicating to the consumer that the topical application of the personal care composition may reduce shaving frequency, improve the ease of hair removal, increase shaving efficiency, make hair softer and finer, make hair less noticeable, slow the re-growth of hair, reduce erythema and irritation to skin, eliminate the risks of cuts and nicks due to shaving, maintain a desired hair style and/or color for a longer period of time, and combinations thereof. The manner in which the communication is conveyed to the consumer is non-limiting. By way of example only, the communication can be effected by known advertisement techniques, such as, television, internet and magazine advertisements. The communication may be a point-of-sale technique, such as, for example, a shelf and/or floor affixed communication. Additionally or alternatively, the communication may take the form of indicia (text, symbols, colors, shades, figures, and the like) disposed in and/or on packaging of the personal care compositions. The communication may comprise images comparing the appearance of the keratinous tissue of a consumer prior to and after use of the composition.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-6

A moisturizing skin cream/lotion is prepared by conventional methods from the following components. When the composition is applied to keratinous tissue in need of hair growth management at least once per day, a reduction in hair growth and/or finer hair may be realized.

| | Example | | | | |
|---|---|---|---|---|---|
| | 1 | 2 | 3 | 4 | 5 |
| Component | wt % | wt % | wt % | wt % | wt % |
| Water | qs | qs | qs | qs | qs |
| Glycerol | 5.0000 | 7.0000 | 7.0000 | 10.000 | 10.000 |
| phenylbenzimidazole sulfonic acid | 1.2500 | 1.2500 | 1.2500 | 1.2500 | 1.2500 |
| disodium EDTA | 0.1000 | 0.1000 | 0.1000 | 0.1000 | 0.1000 |
| Allantoin | 0.2000 | 0.2000 | 0.2000 | 0.2000 | 0.2000 |
| Triethanolamine | 0 | 0 | 0 | 0 | 0.7500 |
| sodium metabisulfite | 0.1000 | 0.1000 | 0.1000 | 0.1000 | 0.1000 |
| BHT | 0.50 | 0.50 | 0.50 | 0.50 | 0.50 |
| titanium dioxide | 0.2500 | 0.4500 | 0.4500 | 0.7500 | 0.5500 |
| Niacinamide | 3.5000 | 3.5000 | 3.5000 | 3.5000 | 3.5000 |
| Dexpanthenol | 0.5000 | 0.5000 | 0.5000 | 0.5000 | 0.5000 |
| palmitoyl-pentapeptide¹ | 0.0004 | 0.0004 | 0.0004 | 0.0004 | 0.0004 |
| C12-C15 alkyl benzoate | 5.00 | 2.5000 | 1.5000 | 2.5000 | 0 |
| caprylic/capric triglyceride | 1.0 | 1.5000 | 1.5000 | 1.5000 | 1.5000 |
| hexamidine | 0.1000 | 0.1000 | 0.3000 | 0.3000 | 0.3000 |
| green tea catechins | 1.000 | 4.000 | 1.000 | 4.000 | 4.000 |
| Octocrylene | 1.0000 | 1.0000 | 1.0000 | 1.0000 | 1.0000 |
| butyl methoxydibenzoylmethane | 2.0000 | 2.0000 | 2.0000 | 2.0000 | 2.0000 |
| cetyl alcohol | 0.5000 | 0.5000 | 0.5000 | 0.5000 | 0.5000 |
| tocopherol acetate | 0.5000 | 0.5000 | 0.5000 | 0.5000 | 0.5000 |
| tocopherol sorbate | 0.5000 | 0.5000 | 0.5000 | 0.5000 | 0.2000 |
| sorbitan stearate/sucrose cocoate | 1.0000 | 1.0000 | 1.0000 | 1.0000 | 1.0000 |
| cetearyl glucoside | 0.5000 | 0.5000 | 0.5000 | 0.5000 | 0.5000 |
| stearyl alcohol | 0.7000 | 0.7000 | 0.7000 | 0.7000 | 0.7000 |
| behenyl alcohol | 0.6000 | 0.6000 | 0.6000 | 0.6000 | 0.6000 |
| ethyl paraben | 0.2000 | 0.2000 | 0.2000 | 0.2000 | 0.2000 |
| propyl paraben | 0.1000 | 0.1000 | 0.1000 | 0.1000 | 0.1000 |
| PEG-100 stearate | 0.1000 | 0.1000 | 0.1000 | 0.1000 | 0.1000 |
| Polymethylsilsesquioxane | 0.2500 | 0.5000 | 1.5000 | 0.5000 | 0.2500 |
| Sepigel ™-305 | 2.000 | 2.000 | 2.5000 | 2.5000 | 3.0000 |
| (polyacrylamide/C13-14 isoparaffin/laureth-7) | | | | | |
| glyceryl dilaurate | 1.000 | 1.000 | 2.5000 | 2.5000 | 2.5000 |
| tetrahydrocurcumin | 0.50 | 0.50 | 1.00 | 1.00 | 1.00 |
| benzyl alcohol | 0.2500 | 0.2500 | 0.2500 | 0.2500 | 0.2500 |
| dimethicone/dimethiconol | 0.5 | 1.0000 | 2.5000 | 0.2500 | 2.0000 |
| Perfume | 0.2000 | 0.2000 | 0.2000 | 0.2000 | 0.2000 |

¹palmitoyl-pentapeptide = palmitoyl-lysine-threonine-threonine-lysine-serine available from Sederma ™.

### Example 6

| Component | % w/w |
|---|---|
| Water | Qs |
| 1,2-Hexanediol | 3.000 |
| Glycerin | 10.000 |
| Hexamidine Diisethionate | 0.100 |
| Sodium Metabisulfite | 0.050 |
| Epigallocatechin Gallate | 2.000 |
| Methylparaben | 0.100 |
| Disodium EDTA | 0.100 |
| Tocopheryl Acetate | 0.250 |
| Isopropyl Isostearate | 1.500 |
| Petrolatum | 2.000 |
| Caprylic/Capric Triglyceride | 0.300 |
| Caprylic/Capric Triglyceride | 0.300 |
| Cetearyl Glucoside, Cetearyl Alcohol | 0.200 |
| Behenyl Alcohol | 0.315 |
| Ethylparaben | 0.200 |
| Propylparaben | 0.100 |
| Tetrahydrocurcumin | 0.500 |
| Cetyl Alcohol | 0.386 |
| Stearyl Alcohol | 0.480 |
| BHT | 0.500 |
| Glyceryl Dilaurate | 2.000 |
| PEG-100 Stearate | 0.200 |
| Aluminum Starch Octenylsuccinate | 0.500 |
| Salicylic acid | 2.000 |
| Urea | 2.000 |
| Polyethylene | 0.500 |
| Polyacrylamide(and)C13-14 | 3.500 |
| Isoparaffin(and)Laureth-7 | |
| Panthenol | 1.000 |
| Benzyl Alcohol | 0.250 |
| Dimethicone and Dimethiconol | 1.000 |

### Examples 7-9

A moisturizing skin cream/lotion for hand and body skin care is prepared by conventional methods from the following components. The coated titanium dioxide is incorporated via the oil phase ingredients whereas the nylon particles and interference pigment are added via the aqueous phase.

| | Example | | |
|---|---|---|---|
| | 7 | 8 | 9 |
| Component | % w/w | % w/w | % w/w |
| Niacinamide | 2.0 | 4.0 | 6.0 |
| Retinyl Propionate | | 0.2 | |
| Panthenol | 1.0 | 2.0 | 0.5 |
| Polyacrylamide & isoparaffin & laureth-7 | 2.0 | 2.25 | 2.25 |
| hexamidine | 0.10 | 0.10 | 0.10 |
| Glycerine | 5.0 | 3.0 | 7.0 |
| Allantoin | 0.2 | 0.05 | 0.1 |
| Aloe vera gel | 0.05 | 0.075 | 0.05 |
| Tocopheryl acetate | 0.75 | 0.5 | 0.5 |
| Cetyl alcohol | 2.0 | 1.0 | 1.25 |
| Stearyl alcohol | 2.0 | 1.0 | 1.25 |
| Behenyl alcohol | 1.0 | 1.0 | 1.25 |
| Dimethicone & dimethiconol | 0.75 | 0.5 | 0.50 |
| Steareth-21 | 0.6 | 0.4 | 0.5 |
| Steareth-2 | 0.1 | 0.08 | 0.03 |
| PPG-15 stearyl ether | 3.0 | 2.0 | 1.00 |
| Isohexadecane | 0 | 7.0 | 5.0 |
| glyceryl dilaurate | 0.50 | 2.50 | 5.00 |
| Isononyl isononanoate | 5.0 | 0 | 0 |
| Dimethicone (350 mm²s⁻¹) | 0.5 | 0.0 | 0.60 |
| Disodium EDTA | 0.10 | 0.10 | 0.10 |
| Nylon 12¹ | 1.5 | 1.0 | 1.1 |
| Titanium Dioxide (and) Mica² | 0.75 | 1.5 | 1.25 |
| BHT | 0.50 | 0.50 | 0.50 |
| Petrolatum | 1.00 | 4.00 | 2.00 |
| Deionised water, fragrance, presevatives | to 100% | to 100% | to 100% |

¹Orgasol ™ 2002 D NAT COS.
²A green interference pigment

### Examples 10-13

An antiperspirant soft solid/cream is prepared by conventional methods from the following components. When applied daily to the underarm region, a reduction in hair growth, softer and/or finer hair, and a reduction in bodily malodor may be realized.

| | Example | | | |
|---|---|---|---|---|
| Component | 10 | 11 | 12 | 13 |
| Al Zr Trichlorohydrex Glycinate | 25.25 | 25.25 | 25.25 | 25.25 |
| (solid) | | | | |
| Dimethicone (10cs) | 5.00 | 5.00 | 5.00 | 5.00 |
| Fully Hydrogenated High Erucic | 5.00 | 5.00 | 5.00 | 5.00 |
| Acid Rapeseed oil (HEAR oil) | | | | |
| glyceryl dilaurate | 2.50 | 2.50 | 1.00 | 1.00 |
| C-18-36 Acid Triglyceride | 1.25 | 1.25 | 1.25 | 1.25 |
| Syncrowax ® HGLC | | | | |
| Perfume | 0.75 | 0.75 | 0.75 | 0.75 |
| hexamidine | 0.100 | 0.30 | 0.10 | 0.30 |
| Calcium Pantothenate (solid) | 0.50 | 0 | 3.50 | 0 |
| BHT | 0.50 | 0.50 | 0.50 | 0.50 |
| Tocopherol Acetate | 0.50 | 0.50 | 0.50 | 0.50 |
| Cyclopentasiloxane | QS | QS | QS | QS |
| Total | 100.00 | 100.00 | 100.00 | 100.00 |

### Example 14

A foundation compact of the present invention comprising cross-linked siloxane elastomer is prepared as follows:

| Ingredient | wt. % |
|---|---|
| TiO2 silicone treated (SAT treated Tronox ™ | 5.25 |
| CR 837 supplied US Cosmetics) | |
| Pigment | 1.23 |
| Talc (silicone treated) (Hydrophobic Talc | 2.36 |
| 9742 supplied by Warner Jenkinson ™) | |
| Glyceryl dilaurate | 2.50 |
| TiO2 - MT100T (micronized TiO2 supplied by | 0.16 |
| Tri-K) | |
| DC245 (cyclomethicone) | 29.26 |
| DC5225C (dimethicone copolyol - 10% active | 0.31 |
| in cyclomethicone) | |
| GE SFE 839 Cross-linked Siloxane Elastomer | 48 |
| Gel¹ | |
| propylparaben (preservative) | 0.10 |
| hexamidine | 0.10 |
| BHT | 0.50 |
| Glycerine | 7.08 |
| Ozokerite Wax | 3.25 |
| Total: | 100.00 |

¹5% Dimethicone/vinyl dimethicone cross-polymer in cyclomethicone

In a suitable vessel equipped with a heating source, the pigments, TiO₂ (micronized and silicone treated), hydrophobic talc, GE SFE 839, cyclomethicone (DC245) and dimethicone copolyol (DC5225C) are mixed until homogeneous and then milled using a Silverson L4RT mixer at 9000 rpms to the desired particle size. Next, the propylparaben and glycerine are added to the above mixture and mixed until homogenous. The mixture is then heated to a temperature of between 85-90° C., at which time the ozokerite wax is added (melted into the mixture) with mixing until the mixture homogenous. The mixture is then poured into a mold and allowed to cool at room temperature. Once cooled, the mixture is incorporated into the appropriate package. The foundation compact is applied to the face to provide color, moisturization and improved feel, and to regulate hair growth and reduce the appearance of dark and/or vellus hair.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govem.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of providing a hair regulation benefit to mammalian keratinous tissue, comprising:
applying to keratinous tissue in need of said benefit a composition comprising at least one hair regulating compound selected from the group consisting of glyceryl dilaurate, apigenin, tetrahydrocurcumin, oleanolic acid, azelaic acid, sulforaphane, canavanine, pyridoxal 5-phosphate, phytic acid, tannic acid, grape seed extract, NG-nitro-L-arginine-methyl ester, benzamidine, sodium butyrate, betulinic acid, polyornithine, polyarginine, fisetin, jasmonates, methyl-jasmonate, cis-jasmone, caffeic acid phenethyl ester, delphinidin, ethyl abietate, esculetin, sorbic acid methyl ester, canaline, N-formyl-methionine, N-formyl-alanine, taurine, palmitoyl carnitine, undecanol, undecylenic acid, rutin, fusidic acid, phenyl pyruvic acid, L-isoleucine, phenyl glycine, silibinin, silymarin, L-ascorbic acid-6-palmitate, N-undecylenoyl-L-phenylalanine, and salts, derivatives and mixtures of any of the foregoing; and a dermatologically-acceptable carrier.

2. The method according to claim 1, wherein the benefit is selected from the group consisting of hair growth regulation, reducing shaving frequency, improving the ease of hair removal, increasing shaving efficiency, making hair softer and finer, making hair less noticeable, slowing the re-growth of hair, reducing erythema and irritation to skin, maintaining a desired hair style for a longer period of time, maintaining a desired hair color for a longer period of time, maintaining a desired facial hair style for a longer period of time, reducing the appearance of pore size, and combinations thereof.

3. The personal care composition of claim 1, further comprising at least one additional compound selected from the group consisting of butylated hydroxytoluene, butylated hydroxyanisole, cetyl pyridinium chloride, a hexamidine compound, a phytosterol, a green tea catechin compound, salicylic acid, urea, ursolic acid, difluoromethylomithine, alpha-methylomithine, 1,8-diaminooctane, bathocuprione, alanosine, arginine, soy extracts, cinnamic acid, cinnamyl anthranilate, mannosamine and salts, derivatives and mixtures of any of the foregoing.

4. The composition of claim 3, wherein the phytosterol is selected from the group consisting of sitosterol, stigmasterol, campesterol, brassicasterol, kinetin, and zeatin, and salts, derivatives and mixtures of any of the foregoing.

5. The composition of claim 3, wherein the green tea catechin compound is selected from the group consisting of epicatechin, epicatechin gallate and epigallocatechin gallate and salts, derivatives and mixtures of any of the foregoing.

6. The composition of claim 1, further comprising at least one additional hair growth regulating compound, selected from the group consisting of resveratrol, wortmannin, palmatine, malonic acid, malic acid, equol, geldanamycin, and combinations thereof.

7. The composition according to claim 1, further comprising at least one chronic skin care active selected from the group consisting of niacinamide, N-acetyl glucosamine, polyacrylamide/C13-14 isoparaffin/laureth-7, carnosine, vitamin E acetate, N-acetyl-D-glucosamine, glucosyl hesperidin, ascorbyl glucoside, palmitoyl-lysine-threonine-threonine-lysine-serine, palmitoyl-lysine-threonine, cetyl betaine, bisabolol, theophylline, dihydroxyacetone, panthenol, butyl methoxydibenzoylmethane, glycyrrhizic acid, glycyrrhetinic acid, ergothioneine, octadecenedioic acid, retinyl propionate, yeast extract, and salts, and derivatives and mixtures of any of the foregoing.

8. The composition according to claim 7, wherein the additional compound is a hexamidine compound and wherein the chronic skin care active is polyacrylamide/C13-14 isoparaffin/laureth-7.

9. The composition according to claim 8, wherein the dermatologically-acceptable carrier comprises hexanediol.

10. A method of providing a hair regulation benefit to mammalian keratinous tissue, comprising:
(a) applying to keratinous tissue in need of said benefit a composition comprising at least one hair regulating compound selected from the group consisting of glyceryl dilaurate, apigenin, tetrahydrocurcumin, oleanolic acid, azelaic acid, sulforaphane, canavanine, pyridoxal 5-phosphate, phytic acid, tannic acid, grape seed extract, NG-nitro-L-arginine-methyl ester, benzamidine, sodium butyrate, betulinic acid, polyornithine, polyarginine, fisetin, jasmonates, methyl-jasmonate, cis-jasmone, caffeic acid phenethyl ester, delphinidin, ethyl abietate, esculetin, sorbic acid methyl ester, canaline, N-formyl-methionine, N-formyl-alanine, taurine, palmitoyl carnitine, undecanol, undecylenic acid, rutin, fusidic acid, phenyl pyruvic acid, L-isoleucine, phenyl glycine, silibinin, silymarin, L-ascorbic acid-6-palmitate, N-undecylenoyl-L-phenylalanine, and salts, derivatives and mixtures of any of the foregoing; and a dermatologically-acceptable carrier; and
(b) delivering energy to the keratinous tissue, wherein the energy is in a form selected from the group consisting of heat, electromagnetic, light, radio-frequency, radio-frequency energy in the absence of light-based energy, mechanical energy, and combinations thereof.

11. The method of claim 10, wherein the mechanical energy is delivered via a mechanical hair removal device selected from the group consisting of blades, razors, epilators, waxes, an electrical powered shaving apparatus, a non-electrical shaving apparatus, and combinations thereof.

12. The method according to claim 10, wherein the benefit is selected from the group consisting of hair growth regulation, reducing shaving frequency, improving the ease of hair removal, increasing shaving efficiency, making hair softer and finer, making hair less noticeable, slowing the re-growth of hair, reducing erythema and irritation to skin, maintaining a desired hair style for a longer period of time, maintaining a desired hair color for a longer period of time, maintaining a desired facial hair style for a longer period of time, reducing the appearance of pore size, and combinations thereof.

13. The composition of claim 10, further comprising at least one additional compound selected from the group consisting of butylated hydroxytoluene, butylated hydroxyanisole, cetyl pyridinium chloride, a hexamidine compound, a phytosterol, a green tea catechin compound, salicylic acid, urea, ursolic acid, difluoromethylomithine, alpha-methylomithine, 1,8-diaminooctane, bathocuprione, alanosine, arginine, soy extracts, cinnamic acid, cinnamyl anthranilate, mannosamine and salts, derivatives and mixtures of any of the foregoing.

14. The composition of claim 13, wherein the phytosterol is selected from the group consisting of sitosterol, stigmasterol, campesterol, brassicasterol, kinetin, and zeatin, and salts, derivatives and mixtures of any of the foregoing.

15. The composition of claim 13, wherein the green tea catechin compound is selected from the group consisting of epicatechin, epicatechin gallate and epigallocatechin gallate and salts, derivatives and mixtures of any of the foregoing.

16. The composition of claim 10, further comprising at least one additional hair growth regulating compound, selected from the group consisting of resveratrol, wortmannin, palmatine, malonic acid, malic acid, equol, geldanamycin, and combinations thereof.

17. The composition according to claim 10, further comprising at least one chronic skin care active selected from the group consisting of niacinamide, N-acetyl glucosamine, polyacrylamide/C13-14 isoparaffin/laureth-7, carnosine, vitamin E acetate, N-acetyl-D-glucosamine, glucosyl hesperidin, ascorbyl glucoside, palmitoyl-lysine-threonine-threonine-lysine-serine, palmitoyl-lysine-threonine, cetyl betaine, bisabolol, theophylline, dihydroxyacetone, panthenol, butyl methoxydibenzoylmethane, glycyrrhizic acid, glycyrrhetinic acid, ergothioneine, octadecenedioic acid, retinyl propionate, yeast extract, and salts, and derivatives and mixtures of any of the foregoing.

18. The composition according to claim 17, wherein the additional compound is a hexamidine compound and wherein the chronic skin care active is polyacrylamide/C13-14 isoparaffin/laureth-7.

19. The composition according to claim 18, wherein the dermatologically-acceptable carrier comprises hexanediol.

20. An article of commerce comprising a composition for regulation of hair growth according to claim 1, and a communication indicating that the composition is useful for providing at least one benefit selected from the group consisting of regulating hair growth, reducing shaving frequency, improving the ease of hair removal, increasing shaving efficiency, making hair softer and finer, making hair less noticeable, slowing the re-growth of hair, reducing erythema and irritation to skin, eliminating the risks of cuts and nicks due to shaving, maintaining a desired hair style for a longer period of time, maintaining a desired hair color for a longer period of time, maintaining a desired facial hair style for a longer period of time, reducing the appearance of pore size, and combinations thereof.
